(19) United States
(12) Patent Application Publication
(10) Pub. No.: US 2025/0327774 A1
(43) Pub. Date: Oct. 23, 2025

(54) QUARTZ CRYSTAL MICROBALANCE DEVICE AND BIOASSAY

(71) Applicant: University of Massachusetts, Westborough, MA (US)

(72) Inventors: Yan Luo, Boston, MA (US); Sheree Pagsuyoin, Acton, MA (US); Calvin Alexander Yu Ng, Lowell, MA (US); Frédéric JJ Chain, Londonderry, NH (US); Jack Regan Lepine, Dracut, MA (US)

(21) Appl. No.: 19/183,179

(22) Filed: Apr. 18, 2025

Related U.S. Application Data
(60) Provisional application No. 63/636,374, filed on Apr. 19, 2024.

(51) Int. Cl. G01N 29/02; G01N 33/543; G01N 33/569

(52) U.S. Cl. CPC G01N 29/022; G01N 33/54373; G01N 33/56911; G01N 33/56983; G01N 2333/01; G01N 2333/165

(57) ABSTRACT

A portable quartz crystal microbalance (QCM) device comprises: a QCM resonator; a first printed circuit board assembly; a user interface; a data transmission means to communicate with a mobile device for data visualization, storage, and processing; a power source; and an enclosure, wherein the QCM resonator comprises a quartz oscillator having at least one lead and at least one characteristic resonant frequency, and is configured to modify the at least one characteristic resonant frequency in response to a quantity of adsorbed material on the quartz oscillator. A bioassay for determining the amount of a biological analyte in a fluid sample with the QCM comprises: selecting a bioprobe that selectively binds the biological analyte; binding the bioprobe onto the surface of the micropillars of resonant material; and detecting a response to adsorption of a biological analyte onto the micropillars of resonant material and bioprobe, measured as a frequency shift.

### CROSS REFERENCE TO RELATED APPLICATIONS

This non-provisional patent application claims the benefit of U.S. Provisional Application No. 63/636,374 filed Apr. 19, 2024, which is incorporated herein by reference in its entirety.

### FEDERAL RESEARCH STATEMENT

This invention was made with U.S. government support under Award ID No. 2329826 awarded by the National Science Foundation. The government has certain rights in the invention.

### BACKGROUND

Quartz crystal microbalances (QCM) have been extensively used in sensing mass loading with extremely high sensitivity (<10 ng/cm²). A QCM device typically consists of a thin disk of AT-cut quartz crystal with circular electrodes patterned on both sides. Due to the piezoelectric properties and crystalline orientation of the quartz crystal, an alternating voltage between the electrodes results in a shear wave within the quartz crystal so that it oscillates at distinct frequencies. The quartz crystal is therefore referred to as a QCM resonator. The resonant frequency and bandwidth depend on the mass of material bound to the quartz crystal. Variations in mass that result from binding different molecules and amounts of molecules on the surface of the QCM resonator can therefore be measured. The quartz crystal surface can be modified with a micropillar structure made from a resonant polymer.

QCM devices are presently desktop instruments used in laboratory settings or other fixed locations. However, many of the expanding number of potential applications of QCM biosensors require enhanced mobility of QCM instruments. Enhanced mobility would enable on-the-fly, in situ, and on-demand testing of samples, without geographical constraints. Hence, there is a growing need for a portable QCM device capable of measuring the resonant frequency of a QCM in a variety of liquid samples, and in a variety of pathogen monitoring applications such as bacterial or virus infectious agents, aquaculture, biomass, environmental protection, and so on. The device should be portable, durable, and sized and light-weight enough to be hand-held. They should also be battery-powered so they are not constrained by availability of external power supply.

Other improvements in QCM devices could expand their ease-of-use and functionality. For example, the QCM device should be versatile and able to detect and quantify a wide variety of analytes. The QCM device should accurately measure the resonate frequency shift of a QCM device with an absolute error of up to 25 Hz. The accuracy of the measurement depends on the drift of the reference clock. For example, the design should achieve 25 Hz accuracy when the stability of the reference clock is +/−2.5 parts in 1,000,000 (ppm). The QCM device should also be capable of data transmission to a mobile device or a laptop computer. The QCM device should also be equipped with appropriate software for control, calibration, and visualization of data. It is also highly desirable that the QCM device can function as a biosensor for the detection of specific microorganisms or biological substances, including pathogens, and measure their concentrations in liquid samples.

### BRIEF DESCRIPTION

A portable quartz crystal microbalance device comprises: a quartz crystal microbalance resonator; a first printed circuit board assembly; a user interface; a data transmission means to communicate with a mobile device for data visualization, storage, and processing; a power source; and an enclosure, wherein the quartz crystal microbalance resonator comprises a quartz oscillator having at least one lead and at least one characteristic resonant frequency, and is configured to modify the at least one characteristic resonant frequency in response to a quantity of adsorbed material on the quartz oscillator.

A bioassay for determining the amount of a biological analyte in a fluid sample with the above quartz crystal microbalance comprises: selecting a bioprobe that selectively binds the biological analyte; binding the bioprobe onto the surface of the micropillars of resonant material; adding the fluid sample to the quartz crystal microbalance device; and detecting a response to adsorption of the biological analyte onto the micropillars of resonant material and bioprobe, measured as a frequency shift.

The above described and other features are exemplified by the following figures and detailed description.

### BRIEF DESCRIPTION OF THE DRAWINGS

The following figures are exemplary embodiments wherein the like elements are numbered alike.

FIG. 1 is a schematic diagram of the portable quartz crystal microbalance (QCM) device, which is in communication with a mobile phone and laptop computer by Bluetooth. The analog-digital printed circuit band (PCB), modular removable QCM cartridge, and handheld enclosure components are indicated.

FIGS. 2A and 2B are photos of a prototype portable QCM device with enclosure and removable QCM cartridge removed from the device (FIG. 2A) and inserted into the device (FIG. 2B).

FIG. 3 is a schematic diagram of the modular removable QCM cartridge including exploded (left) and assembled (right) view. In the exploded view (left), the modular removable QCM comprises a reservoir for liquid samples and the QCM resonator (A), a sealing gasket (B) to prevent leaks, and a miniature PCB with contact pads on both sides (C). The three components assembled are depicted schematically (right). Ingress channels indicated by black dots on the top of component (A) and addition of a liquid sample by a syringe are also depicted.

FIG. 4 depicts partially disassembled views of the modular removable QCM cartridge, with (a) being the reservoir for the fluid sample and channels for fluid sample ingress and egress, (b) being the QCM resonator, (c) being the gasket to prevent leakage of the fluid sample, and (d) being electrical contacts between the QCM resonator and the analog-digital PCB.

FIG. 5 is the schematic of a circuit for QCM signal processing. The circuit comprises the oscillating circuit containing the QCM and a transconductance amplifier, a variable gain amplifier, a digital proportional-integral (PI) controller, and a rectifier. The circuit generates a high frequency output signal (HF-OUT).

FIG. 6 is circuit diagram for digitization, frequency counting, and data transmission of the HF-OUT analog signal from the QCM signal processing circuit. The circuit includes Schmitt trigger with a counter integrated circuit (IC), a high-precision reference clock with a counter IC, multiplexer, analog temperature reading, and microcontroller, and outputs signals to a mobile phone or laptop via Bluetooth.

FIGS. 7A and 7B illustrate alternative examples of data acquisition and visualization on a mobile user interface. In FIG. 7A, the mobile user interface provides the current frequency reading and geographical location of the sensor. In FIG. 7B, the mobile user interface provides time-dependent frequency and temperature data.

FIG. 8 is a schematic diagram of a sensor network of QCM devices (wastewater sensors) that transfers sensor data to a cloud server via a cellular network. The cloud server supports remote access (data queries) initiated by laptop and a real-time monitoring dashboard.

FIG. 9 a screenshot of an oscilloscope display showing that the circuit captures a stable 10.0062 MHz resonance frequency of a QCM based biosensor.

FIGS. 10A and 10B are photos of the analog-digital PCB (Revision 3) pre-assembly (FIG. 10A) and post-assembly (FIG. 10B).

FIG. 11 depicts graphically a frequency shift when the QCM device is tested with a sample of white spot syndrome virus (WSSV). T₁ represents a baseline frequency of 9,982,320 Hz, T2 represents a region of shifting frequency when the sample is introduced, and T₃ represents a new baseline frequency of 9,979,374 Hz after sample introduction.

FIG. 12 is schematic of a white spot syndrome virus (WSSV) detection process in the bioassay showing complementarity binding of DNA/RNA of WSSV and an oligonucleotide bioprobe complementary to the DNA/RNA of WSSV adsorbed onto a PMMA micropillar surface of the QCM resonator.

### DETAILED DESCRIPTION

The present inventors have designed a QCM device that is portable, durable, and sized and light-weight enough to be hand-held. The QCM device has other features that likewise expand their ease-of-use and functionality. Advantageously, the QCM device is versatile and capable of measuring a variety of analytes, by using modular removable QCM cartridges, each configured for detection and measurement of different analytes. The QCM device measures the resonate frequency shift of an analyte with high accuracy. In some embodiments, the QCM device accurately measures the resonate frequency shift of a QCM device with an absolute error of up to 25 Hz. The accuracy of the measurement depends on the drift of the reference clock. For example, the design achieves 25 Hz accuracy when the stability of the reference clock is +/−2.5 parts in 1,000,000 (ppm). The QCM device is also equipped to measure the temperature of the test sample to correlate sample temperature with frequency shift. Advantageously, the QCM device is also capable of data transmission to a mobile device or a laptop computer, including through a cellular network. The QCM device is also equipped with software for control, calibration, and visualization of data. The QCM device is ideally suited for the measurement of biological substances and bacterial or virus infectious agents.

The QCM device comprises: a QCM resonator; a first printed circuit board (PCB) assembly; a user interface; a data transmission means to communicate with a mobile device for data visualization, storage, and processing; a power source; and an enclosure, wherein the QCM resonator comprises a quartz oscillator having at least one lead and at least one characteristic resonant frequency, and is configured to modify the at least one characteristic resonant frequency in response to a quantity of adsorbed material on the quartz oscillator. The QCM resonator can be contained in a modular removable QCM cartridge that plugs into the device.

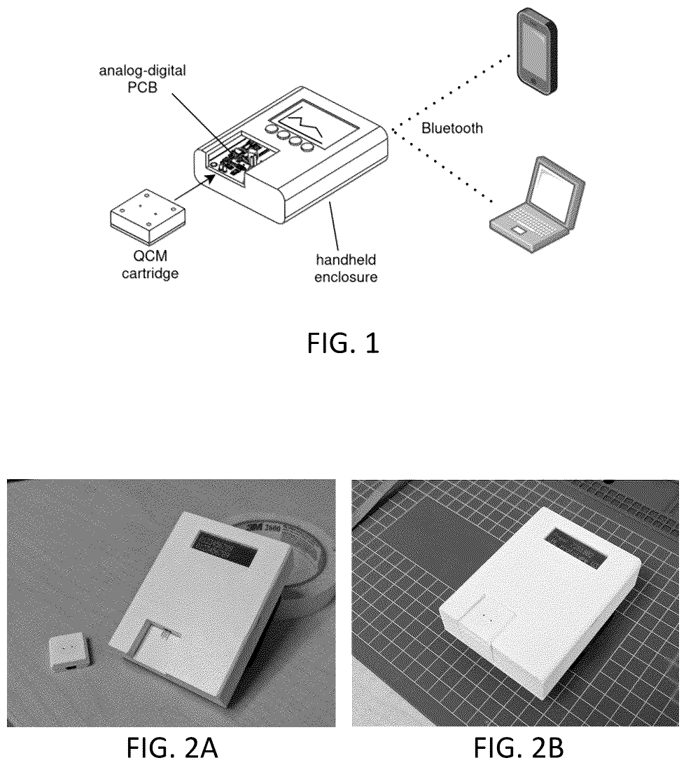

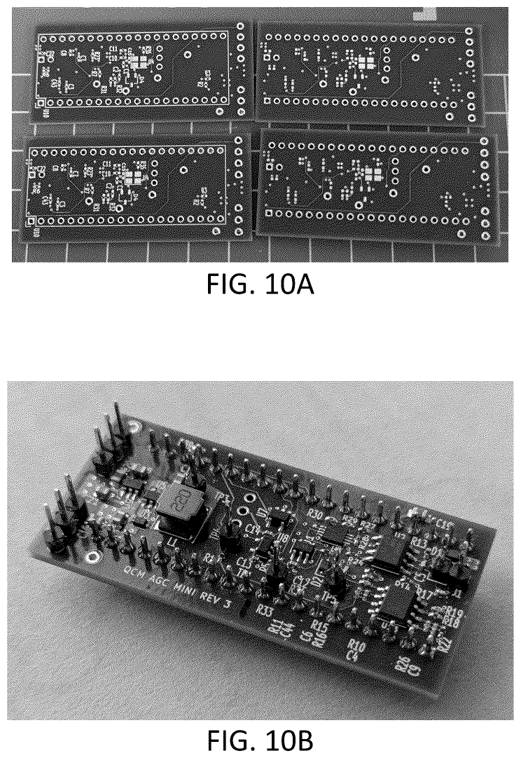

FIG. 1 illustrates the overall architecture of the device. The enclosure includes a guided track through which a removable QCM cartridge can be slid in place such that the cartridge leads make contact to the electrical contact pads of the first PCB. The first PCB is an analog-to-digital PCB. It contains analog and digital circuitry to amplify and filter signals from the QCM resonator such that a stable resonant frequency can be detected and measured. FIG. 9 is a screenshot of an oscilloscope display showing that the digital counting system does capture a stable 10.0062 MHz resonance frequency of the QCM device. FIGS. 10A and 10B are photos of the first (analog-to-digital) PCB (Revision 3) pre-assembly (FIG. 10A) and post-assembly (FIG. 10B).

The QCM resonator can have a micropillar structure. Thus, in some embodiments, the quartz crystal microbalance resonator comprises: a quartz oscillator having a surface and having at least one lead, and a plurality of micropillars of a resonant material, each of the micropillars having a diameter, a height, and a spacing, together forming a patterned array of micropillars, and a residual layer situated between the plurality of micropillars and said quartz oscillator, wherein: the resonant material is a polymer, the plurality of micropillars is in mechanical communication with said surface of the quartz oscillator through the residual layer, and the quartz crystal microbalance resonator has at least one characteristic resonant frequency and is configured to modify said at least one characteristic resonant frequency in response to a quantity of adsorbed material on said plurality of micropillars. In some embodiments, the resonant polymer is poly(methyl methacrylate) (PMMA). Examples of QCM resonators having a micropillar structure, and designated QCM-P, are disclosed in U.S. Pat. No. 10,268,114 B2, incorporated by reference in its entirety herein. General procedures for the preparation of QCM-P resonators is described therein.

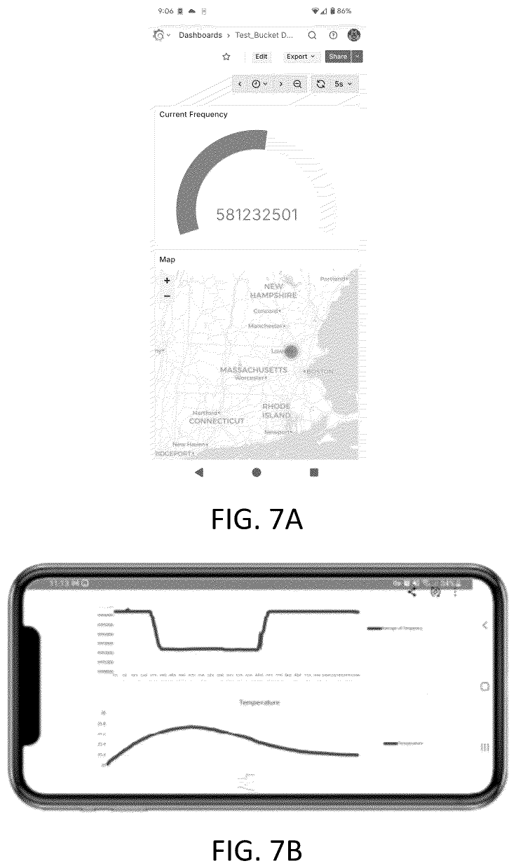

A problem with QCM devices is that the resonant frequency can be sensitive to temperature fluctuations. Advantageously, the first PCB can be configured to measure the temperature of the sample to enable compensation for frequency shifts due to temperature variations. In some embodiments, the first PCB further comprises a temperature sensor to enable compensation for these frequency shifts due to temperature variations. The QCM device is thereby configured to transmit both frequency and temperature data to a mobile device. The measurement data including frequency and temperature are transmitted to a smartphone or a laptop computer for storage, processing and visualization. The user interface comprises buttons and an LCD display. The system can be powered by a battery, for example a 9-V battery. Several features of the QCM device are illustrated schematically in FIG. 1. The QCM device is in communication with a mobile phone and laptop computer by Bluetooth. The first analog-digital PCB, modular removable QCM cartridge, and handheld enclosure components are all indicated. FIGS. 2A and 2B are photos of a prototype portable QCM device with enclosure and modular removable QCM cartridge removed (FIG. 2A) and fully assembled (FIG. 2B). FIGS. 7A and 7B illustrate data acquisition and visualization on a mobile user interface. The user interface of FIG. 7A depicts current frequency reading and geographical location of the sensor, while the user interface of FIG. 7B depicts time-dependent frequency and temperature data.

Advantageously, the QCM device is portable. In some embodiments, the portable QCM device is sized and configured to be hand-held. QCM devices are presently desktop instruments used in laboratory settings or other fixed locations. However, many of the expanding number of potential applications of QCM devices configured as biosensors require enhanced mobility. Advantageously, the present hand-held QCM device enables on-the-fly, in situ, and on-demand testing of samples without geographical constraints. Thus, in some embodiments, the portable QCM device occupies a volume corresponding to less than or equal to 4 in.×3 in.×1 in. and greater than or equal to 2 in.×2 in.×1 in. In some embodiments, the portable QCM device measures about 2 in×about 2 in.×about 1 in. The portable QCM device can weigh about 100 to about 1,000 grams, specifically about 100 to 200 grams. In some embodiments, the portable QCM device weighs about 200 grams.

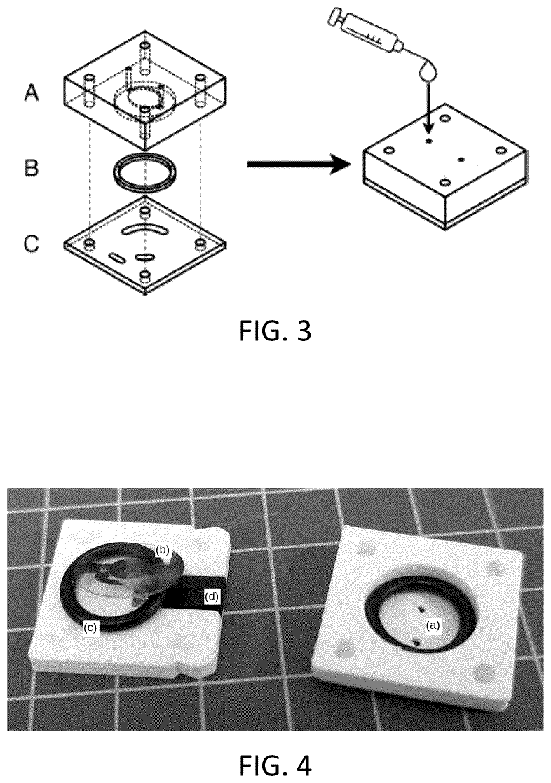

The QCM device includes a modular removable QCM cartridge, which can be configured as a biosensor. Biosensors inherently have a limited lifetime and are configured for detection of specific analytes. Advantageously, the capability of replacing the biosensor supports a multitude of different field applications, saves the cost of replacing the entire QCM device for different analytes, and simplifies maintenance. In this way, the first PCB and associated software are retained and reused for sample testing with a new biosensor. For this purpose, the QCM device includes a modular removable QCM cartridge that can serve as a replaceable biosensor component. It can be inserted into, or removed from, the QCM device as needed. As illustrated schematically in FIG. 3, the modular removable cartridge includes three layers: (A) a reservoir that can hold liquid samples and the QCM resonator; (B) a sealing gasket to prevent leaks; and (C) a miniature PCB (second PCB) with contact pads on both sides (the top side to a metal lead of the QCM resonator, and the bottom side to the contact leads of the main PCB (first PCB). The reservoir of layer (A) is enclosed and has ingress and egress channels for input and removal of liquid samples, respectively. Thus, the modular removable QCM cartridge comprises at least one ingress channel for fluid sample input and at least one egress channel for fluid sample output, and the ingress and egress channels are both accessible when the cartridge is plugged into the device. A syringe can be used to inject a sample containing analyte into the reservoir via an ingress channel as depicted in FIG. 3 (right). After one measurement procedure, a syringe can be used to push deionized water through the reservoir for cleaning in preparation for the next measurement. Between layers (A) and (C), there is a gasket that sits in a recessed channel such that the reservoir is sealed against leakage from the reservoir to the electronics of the removable QCM (first and second PCBs). If necessary, pressure can be applied to (A) and (C) to achieve a perfect seal against leakage. The three layers are compacted together and enclosed in the cartridge, which has two openings on the top surface, i.e. the ingress and egress channels of the reservoir, and three metal contacts protruding at the bottom for making contact to the first PCB.

FIG. 4 provides partially disassembled views of a modular removable QCM cartridge prototype, with (a) being the reservoir for the fluid sample and channels for fluid sample ingress and egress, (b) being the QCM resonator, (c) being the gasket to prevent leakage of the fluid sample, and (d) being the electrical contacts between the QCM resonator and the first analog-digital PCB. Thus, the modular removable QCM cartridge comprises: a reservoir that can hold a fluid sample and the QCM resonator; a gasket to prevent leakage of the fluid sample; and a second PCB having contact pads on the top side and on the bottom side, wherein the contact pad on the top side is configured to connect to the at least one lead of the QCM oscillator, and the contact pad on the bottom side is configured to connect to at least one lead of the first PCB.

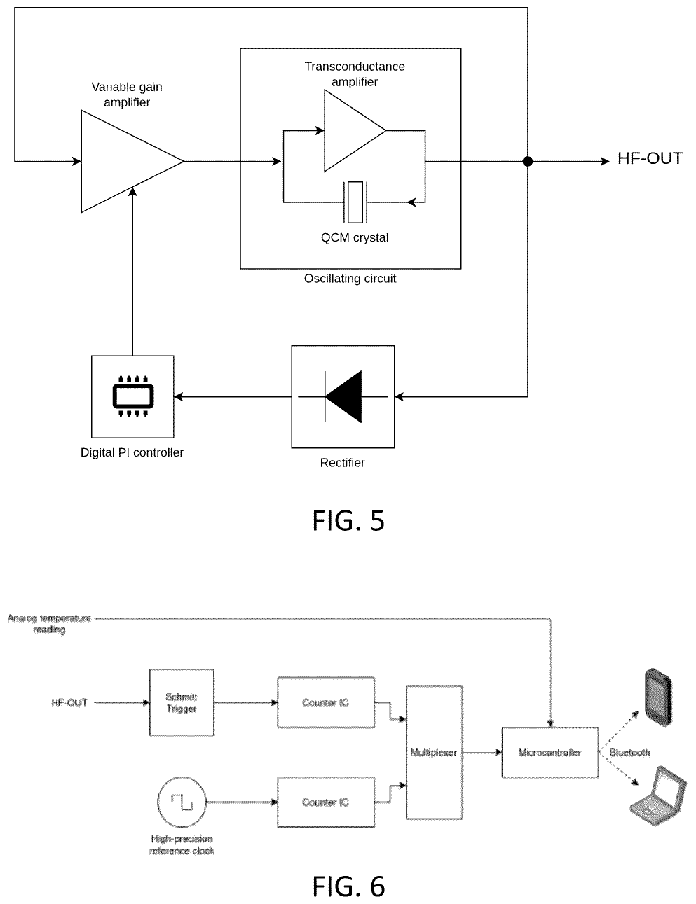

The first PCB of the QCM device comprises a novel compact oscillation circuit for the processing of signals from the QCM resonator. This circuit normalizes the amplitude of an oscillator, i.e. the quartz oscillator, and allows lower Q-factor oscillations to be detectable. FIG. 5 is a schematic illustrating the major components of the circuit for QCM signal processing. The circuit comprises the oscillating circuit containing the QCM and a transconductance amplifier, containing the QCM and transconductance amplifier, a variable gain amplifier, a digital proportional-integral (PI) controller, and a rectifier. The circuit generates a high frequency output signal (HF-OUT), which is an amplified AC signal with can have an amplitude of about 400 mV. A variable gain amplifier is included and is under digital PI control with a microcontroller (or an FPGA chip). In addition, the rectifier can be replaced by a simpler circuit consisting of a diode, capacitor, and resistor. The HF-OUT frequency represents the resonate frequency of the QCM resonator (with or without test samples applied). Thus, the first PCB comprises a compact oscillation circuit comprising a variable gain amplifier, a digital proportion-integral (PI) controller, and a rectifier, wherein the compact oscillation circuit is configured to normalize amplitude of the quartz oscillator so that lower Q-factor oscillations are detectable and to provide high frequency analog output.

The QCM device consists of a digital circuitry that analyzes the signal HF-OUT from the compact oscillation circuit to accurately measure the frequency through a digital counting scheme implemented with counter integrated circuits (ICs), a multiplexer IC, and an off-the-shelf microcontroller module with data communication capabilities. The microcontroller can be, for example, a Raspberry Pi Pico Wireless microcontroller. Temperature readings can be converted to digital values with the microcontroller. The QCM device data (frequency and temperature) can be transmitted to a mobile device (a smartphone or a laptop computer) through Bluetooth. FIG. 6 is a circuit diagram for digitization, frequency counting, and data transmission of the HF-OUT analog signal from the QCM signal processing circuit. The circuit includes Schmitt trigger with a counter integrated circuit (IC), a high-precision reference clock with a counter IC, multiplexer, analog temperature reading, and microcontroller, and outputs signals to a mobile phone or laptop via Bluetooth. Thus, the QCM device further comprises a digital counting system comprising a Schmitt trigger, a high-precision analog clock, counter integrated circuits, a multiplexer, and a microcontroller, wherein the digital counting system is configured to measure frequency of the high frequency analog output from the compact oscillation circuit. FIG. 9 is a screenshot of an oscilloscope display showing that the digital counting system captures a stable 10.0062 MHz resonance frequency of the QCM device.

The QCM device utilizes calibration methods needed to calibrate the QCM resonator based on ambient environmental parameters, for example temperature and optionally humidity. The methods are therefore supported by temperature and optionally humidity sensors on the first PCB and by algorithms executed on the microcontroller of the digital counting system. Software is utilized to support data acquisition and visualization as well as the control and maintenance of the QCM device. In the digital counting system, the raw time series data (frequency, temperature, and optionally humidity) denoted as (R) are acquired and processed on the microcontroller using a customized algorithm. This algorithm will produce a numerical value (L) that represents the presence and/or load level of the target analyte to be detected and measured in the fluid sample. The raw data (R) and the output values (L) are displayed on an LCD built into the QCM device or visualized on the screen of a connected mobile device. FIG. 7 illustrates data acquisition and visualization on a mobile user interface. The interface, which can be a mobile phone or a laptop computer, provides the current frequency reading and geographical location of the QCM device. FIGS. 7A and 7B illustrate alternative examples of data acquisition and visualization on a mobile user interface. In FIG. 7A, the mobile user interface provides the current frequency reading and geographical location of the sensor. In FIG. 7B, the mobile user interface provides time-dependent frequency and temperature data in graphical form.

The control and maintenance of the system is operated through software functions implemented on the connected mobile device. The control functions include power on/off, test start/stop, set customized detection parameters (such as temperature compensation, readout delay, etc.). The maintenance function can put the system into “service” mode to allow advanced diagnosis and troubleshooting. The deployed devices form a sensor network that transfers sensor data to the cloud server, which in turn supports data query and a real-time monitoring dashboard.

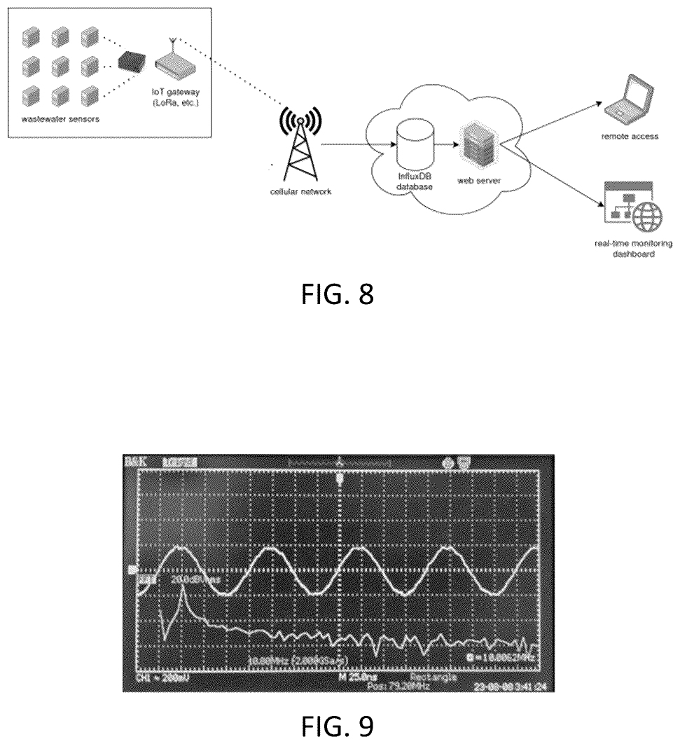

FIG. 8 is a schematic diagram of a sensor network of QCM devices (in this case, wastewater sensors) connected by an internet of things (IoT) gateway that transfers sensor data to a cloud server via a cellular network. The IoT gateway is configured to transmit data, for example by Long Range (LoRa) radio communication. The cloud server includes a time series database (TSDB) and a web server. The TSDB can be, for example, InfluxDB, developed by InfluxData. This system supports remote access (data queries) and a real-time monitoring dashboard accessible on connected mobile devices. Thus, in some embodiments, a system comprises: at least one portable quartz crystal microbalance device as disclosed herein; an internet of things gateway; a cellular network; a cloud-based time series database; a cloud-based web server; and a mobile device; wherein the mobile device is in communication with the at least one portable quartz crystal microbalance device for data visualization, storage, and processing. In some embodiments, the system comprises a network of at least two portable QCM devices. A system composed of a network of nine wastewater sensors is illustrated schematically in FIG. 8.

The QCM devices can be used as biosensors to detect specific microorganisms or biological substances and determination of their concentration in a liquid sample. The microorganisms can be a unicellular organism. The microorganisms can also be pathogenic microorganisms. Thus, a bioassay for determining the amount of a biological analyte in a fluid sample with the quartz crystal microbalance device disclosed herein comprises: selecting a bioprobe that binds the biological analyte; binding the bioprobe onto the surface of the micropillars of resonant material; adding the fluid sample to the quartz crystal microbalance device; and detecting a response to adsorption of the biological analyte onto the micropillars of resonant material and bioprobe, measured as a frequency shift.

As mentioned above, fabrication of the QCM resonator is disclosed in U.S. Pat. No. 10,268,114 B2, incorporated by reference in its entirety herein. The resonant material used in the examples listed in the Table was made from polymethyl methacrylate (PMMA) fabricated into a 17-μm micropillar surface. The resonant material was sequentially: pre-treated with oxygen plasma for two minutes; treated with 0.6% (v/v) aqueous polyethyleneimine; washed with water; and dried under a stream of nitrogen. Then the bioprobes were adsorbed onto the micropillar surface by simple immersion of the QCM resonator in solutions of the bioprobes for two hours, rinsing with phosphate buffer, blocking any free (i.e., no bioprobe attached) micropillar surface with 1% (v/v) aqueous bovine serum albumin in phosphate buffer, and washing with water.

In the bioassay, the micropillars have a surface layer of a bioprobe that selectively binds a biological analyte. Also in the bioassay, the quartz crystal microbalance resonator comprising micropillars and the bioprobe is contained in the portable quartz crystal microbalance device disclosed herein. In the bioassay, the fluid sample comprises a biofluid. The biofluid can comprise a biologically active molecule or a unicellular organism. For example, the unicellular organism can be a virus, a protozoan, or a bacterium. In some embodiments, the organism is a pathogen, and the bioassay is configured for the detection of pathogens. The biological analyte can be inside a cell membrane. Thus, in some embodiments, the fluid sample containing the biological analyte is mixed with lysis buffer prior to input of the fluid sample into the QCM device. This serves to break down cell membranes and release analytes for binding to the bioprobe. The lysis buffer comprises an inorganic base, for example sodium hydroxide and a surfactant, for example sodium dodecyl sulfate.

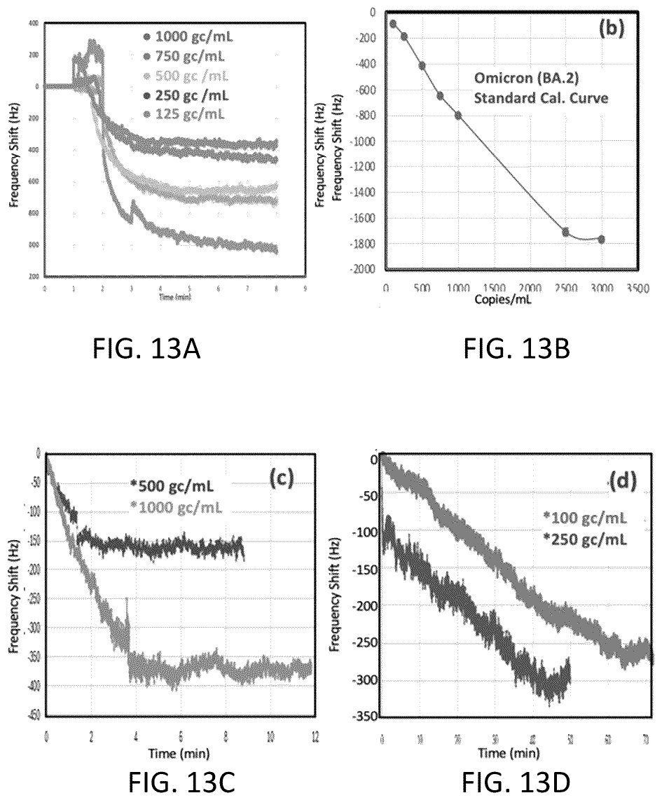

The analyte that binds to the bioprobe can be DNA or RNA of a gene of a microorganism. For example, FIG. 12 is schematic of an E. coli detection process in an E. coli bioassay showing complementarity binding of a bioprobe attached to a PMMA micropillar surface to DNA/RNA from E. coli. A non-limited listing of exemplary pathogens that can be detected by the bioassay and the bioprobes utilized to detect the pathogens is given in the Table.

TABLE
Exemplary Pathogens and Associated Bioprobes

| Pathogen | Bioprobe |
|---|---|
| SARS-CoV-2 | Oligonucleotide complementary to the nucleocapsid (N1) gene |
| Omicron BA.2 (Variant) | Oligonucleotide complementary to the spike (S) gene |
| MERS-CoV | Oligonucleotide complementary to the nucleocapsid (N) gene |
| Vibrio parahaemolyticus | Oligonucleotide complementary to the thermostable direct |
| | hemolysin (tdh) gene |
| White spot syndrome virus | Oligonucleotide complementary to the (VP664) gene that |
| (WSSV) | encodes the major capsid protein VP664 |

The bioassay is applicable to a wide variety of pathogens and complementary bioprobe-biological analyte pairs. In some embodiments, the analyte is the nucleocapsid (N1) gene of SARS-CoV-2 and the bioprobe the oligonucleotide complementary to the nucleocapsid (N1) gene; the analyte is the spike(S) gene of Omicron BA.2 (variant) and the bioprobe is the oligonucleotide complementary to the spike(S) gene; the analyte is the nucleocapsid (N) gene of MERS-CoV and the bioprobe is oligonucleotide complementary to the nucleocapsid (N) gene; the analyte is the thermostable direct hemolysin (tdh) gene of Vibrio parahaemolyticus and the bioprobe is the oligonucleotide complementary to the thermostable direct hemolysin (tdh) gene; or the analyte is the nucleocapsid (VP664) gene that encodes the nucleocapsid protein (VP664) of white spot syndrome virus, and the bioprobe is an oligonucleotide complementary to the nucleocapsid (VP664) gene.

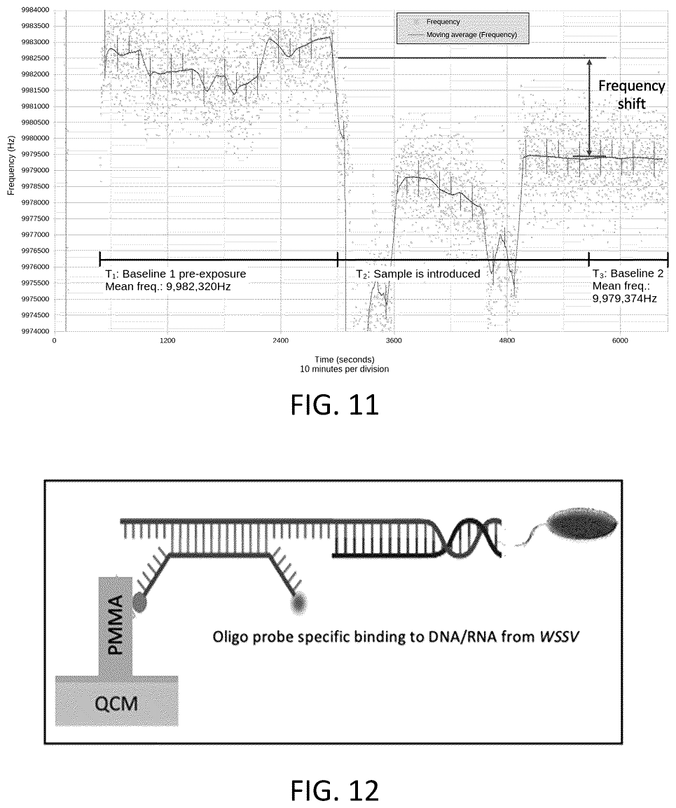

FIG. 11 depicts graphically the frequency shift obtained when the bioassay is designed to detect white spot syndrome virus (WSSV) using a QCM device having an oligonucleotide complementary to the nucleocapsid (VP664) gene of WSSV adsorbed onto micropillars of PMMA in the QCM resonator. T₁ represents a baseline frequency of 9,982,320 Hz, T₂ represents a region of shifting frequency when the liquid sample is introduced, and T₃ represents a new baseline frequency of 9,979,374 Hz after sample introduction.

This disclosure further encompasses the following numbered embodiments and their interrelationships.

Embodiment 1. A portable quartz crystal microbalance device comprises: a quartz crystal microbalance resonator; a first printed circuit board assembly; a user interface; a data transmission means to communicate with a mobile device for data visualization, storage, and processing; a power source; and an enclosure, wherein the quartz crystal microbalance resonator comprises a quartz oscillator having at least one lead and at least one characteristic resonant frequency, and is configured to modify the at least one characteristic resonant frequency in response to a quantity of adsorbed material on the quartz oscillator.

Embodiment 2. The portable quartz crystal microbalance device of Embodiment 1, wherein the quartz crystal microbalance resonator comprises: a quartz oscillator having a surface, at least one lead, and a plurality of micropillars of a resonant material, each of the micropillars having a diameter, a height, and a spacing, together forming a patterned array of micropillars, and a residual layer situated between the plurality of micropillars and said quartz oscillator, wherein: the resonant material is a polymer, the plurality of micropillars is in mechanical communication with said surface of the quartz oscillator through the residual layer and the quartz crystal microbalance resonator has at least one characteristic resonant frequency and is configured to modify said at least one characteristic resonant frequency in response to a quantity of adsorbed material on said plurality of micropillars.

Embodiment 3. The portable quartz crystal microbalance device of Embodiment 1, wherein the first PCB is an analog-to-digital PCB.

Embodiment 4. The portable quartz crystal microbalance device of Embodiment 3, wherein the first PCB comprises a compact oscillation circuit comprising: a variable gain amplifier; a digital proportion-integral (PI) controller; and a rectifier; wherein the compact oscillation circuit is configured to normalize amplitude of the quartz oscillator so that lower Q-factor oscillations are detectable and to provide high frequency analog output.

Embodiment 5. The portable quartz crystal microbalance device of Embodiment 4, further comprising a digital counting system comprising a Schmitt trigger, a high-precision analog clock, counter integrated circuits, a multiplexer, and a microcontroller, wherein the digital counting system is configured to measure frequency of the high frequency analog output.

Embodiment 6. The portable quartz crystal microbalance device of Embodiment 3, wherein the first printed circuit board further comprises a temperature sensor to enable compensation for frequency shifts due to temperature variations.

Embodiment 7. The portable quartz crystal microbalance device of Embodiment 1, configured to transmit frequency and temperature data to a mobile device.

Embodiment 8. The portable quartz crystal microbalance device of Embodiment 1, wherein the quartz crystal microbalance resonator is contained in a modular removable quartz crystal microbalance cartridge that plugs into the device.

Embodiment 9. The portable quartz crystal microbalance device of Embodiment 8, wherein the modular removable quartz crystal microbalance cartridge comprises at least one ingress channel for fluid sample input and at least one egress channel for fluid sample output, and where the ingress and egress channels are both accessible when the cartridge is plugged into the device.

Embodiment 10. The portable quartz crystal microbalance device of Embodiment 8, wherein the modular removable quartz crystal microbalance cartridge comprises: a reservoir that can hold a fluid sample and the quartz crystal microbalance resonator; a gasket to prevent leakage of the fluid sample; and a second printed circuit board having contact pads on the top side and on the bottom side, wherein the contact pad on the top side is configured to connect to the at least one lead of the quartz crystal microbalance oscillator, and the contact pad on the bottom side is configured to connect to at least one lead of the first printed circuit board.

Embodiment 11. The portable quartz crystal microbalance device of Embodiment 1, wherein the device is sized and configured to be hand-held.

Embodiment 12. A system comprising: the at least one portable quartz crystal microbalance device of Embodiment 1; an internet of things gateway; a cellular network; a cloud-based time series database; a cloud-based web server; and a mobile device; wherein the mobile device is in communication with the at least one portable quartz crystal microbalance device for control of the quartz crystal microbalance device and for data visualization, storage, and processing.

Embodiment 13. The system of Embodiment 12, comprising a network of at least two of the portable crystal microbalance devices.

Embodiment 14. A bioassay for determining the amount of a biological analyte in a fluid sample with the quartz crystal microbalance device of Embodiment 2, comprising: selecting a bioprobe that selectively binds the biological analyte; binding the bioprobe onto the surface of the micropillars of resonant material; adding the fluid sample to the quartz crystal microbalance device; and detecting a response to adsorption of the biological analyte onto the micropillars of resonant material and bioprobe, measured as a frequency shift.

Embodiment 15. The bioassay of Embodiment 14, configured for detection of pathogens.

Embodiment 16. The bioassay of Embodiment 14, wherein the fluid sample is mixed with lysis buffer prior to input of the fluid sample into the quartz crystal microbalance device.

Embodiment 17. The bioassay of Embodiment 14, wherein: the analyte is the nucleocapsid (N1) gene of SARS-CoV-2 and the bioprobe is the oligonucleotide complementary to the nucleocapsid (N1) gene; the analyte is the spike(S) gene of Omicron BA.2 (variant) and the bioprobe is the oligonucleotide complementary to the spike(S) gene; the analyte is the nucleocapsid (N) gene of MERS-CoV and the bioprobe is oligonucleotide complementary to the nucleocapsid (N) gene; the analyte is the thermostable direct hemolysin (tdh) gene of Vibrio parahaemolyticus and the bioprobe is the oligonucleotide complementary to the thermostable direct hemolysin (tdh) gene; or the analyte is the nucleocapsid (VP664) gene that encodes the nucleocapsid protein (VP664) of white spot syndrome virus, and the bioprobe is an oligonucleotide complementary to the nucleocapsid (VP664) gene.

The devices and bioassays disclosed herein can alternatively comprise, consist of, or consist essentially of, any appropriate materials, steps, or components herein disclosed. The devices and bioassays can additionally, or alternatively, be formulated so as to be devoid, or substantially free, of any materials (or species), steps, or components, that are otherwise not necessary to the achievement of the function or objectives of the devices and bioassays.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

What is claimed is:

1. A portable quartz crystal microbalance device comprising:
a quartz crystal microbalance resonator;
a first printed circuit board assembly;
a user interface;
a data transmission means to communicate with a mobile device for data visualization, storage, and processing;
a power source; and
an enclosure,
wherein the quartz crystal microbalance resonator comprises a quartz oscillator having at least one lead and at least one characteristic resonant frequency, and is configured to modify the at least one characteristic resonant frequency in response to a quantity of adsorbed material on the quartz oscillator.

2. The portable quartz crystal microbalance device of claim 1, wherein the quartz crystal microbalance resonator comprises:
a quartz oscillator having a surface, at least one lead, and a plurality of micropillars of a resonant material, each of the micropillars having a diameter, a height, and a spacing, together forming a patterned array of micropillars, and
a residual layer situated between the plurality of micropillars and said quartz oscillator,
wherein:
the resonant material is a polymer,
the plurality of micropillars is in mechanical communication with said surface of the quartz oscillator through the residual layer, and
the quartz crystal microbalance resonator has at least one characteristic resonant frequency and is configured to modify said at least one characteristic resonant frequency in response to a quantity of adsorbed material on said plurality of micropillars.

3. The portable quartz crystal microbalance device of claim 1, wherein the first PCB is an analog-to-digital PCB.

4. The portable quartz crystal microbalance device of claim 3, wherein the first PCB comprises a compact oscillation circuit comprising:
a variable gain amplifier;
a digital proportion-integral (PI) controller; and
a rectifier;
wherein the compact oscillation circuit is configured to normalize amplitude of the quartz oscillator so that lower Q-factor oscillations are detectable and to provide high frequency analog output.

5. The portable quartz crystal microbalance device of claim 4, further comprising a digital counting system comprising a Schmitt trigger, a high-precision analog clock, counter integrated circuits, a multiplexer, and a microcontroller, wherein the digital counting system is configured to measure frequency of the high frequency analog output.

6. The portable quartz crystal microbalance device of claim 3, wherein the first printed circuit board further comprises a temperature sensor to enable compensation for frequency shifts due to temperature variations.

7. The portable quartz crystal microbalance device of claim 1, configured to transmit frequency and temperature data to a mobile device.

8. The portable quartz crystal microbalance device of claim 1, wherein the quartz crystal microbalance resonator is contained in a modular removable quartz crystal microbalance cartridge that plugs into the device.

9. The portable quartz crystal microbalance device of claim 8, wherein the modular removable quartz crystal microbalance cartridge comprises at least one ingress channel for fluid sample input and at least one egress channel for fluid sample output, and where the ingress and egress channels are both accessible when the cartridge is plugged into the device.

10. The portable quartz crystal microbalance device of claim 8, wherein the modular removable quartz crystal microbalance cartridge comprises:
a reservoir that can hold a fluid sample and the quartz crystal microbalance resonator;
a gasket to prevent leakage of the fluid sample; and
a second printed circuit board having contact pads on the top side and on the bottom side, wherein the contact pad on the top side is configured to connect to the at least one lead of the quartz crystal microbalance oscillator, and the contact pad on the bottom side is configured to connect to at least one lead of the first printed circuit board.

11. The portable quartz crystal microbalance device of claim 1, wherein the device is sized and configured to be hand-held.

12. A system comprising:
the at least one portable quartz crystal microbalance device of claim 1;
an internet of things gateway;
a cellular network;
a cloud-based time series database;
a cloud-based web server; and
a mobile device;
wherein the mobile device is in communication with the at least one portable quartz crystal microbalance device for control of the quartz crystal microbalance device and for data visualization, storage, and processing.

13. The system of claim 12, comprising a network of at least two of the portable crystal microbalance devices.

14. A bioassay for determining the amount of a biological analyte in a fluid sample with the quartz crystal microbalance device of claim 2, comprising:
selecting a bioprobe that selectively binds the biological analyte;
binding the bioprobe onto the surface of the micropillars of resonant material;
adding the fluid sample to the quartz crystal microbalance device; and
detecting a response to adsorption of the biological analyte onto the micropillars of resonant material and bioprobe, measured as a frequency shift.

15. The bioassay of claim 14, configured for detection of pathogens.

16. The bioassay of claim 14, wherein the fluid sample is mixed with lysis buffer prior to input of the fluid sample into the quartz crystal microbalance device.

17. The bioassay of claim 14, wherein:
the analyte is the nucleocapsid (N1) gene of SARS-CoV-2 and the bioprobe is the oligonucleotide complementary to the nucleocapsid (N1) gene;
the analyte is the spike(S) gene of Omicron BA.2 (variant) and the bioprobe is the oligonucleotide complementary to the spike(S) gene;
the analyte is the nucleocapsid (N) gene of MERS-CoV and the bioprobe is oligonucleotide complementary to the nucleocapsid (N) gene;
the analyte is the thermostable direct hemolysin (tdh) gene of Vibrio parahaemolyticus and the bioprobe is the oligonucleotide complementary to the thermostable direct hemolysin (tdh) gene; or
the analyte is the nucleocapsid (VP664) gene that encodes the nucleocapsid protein (VP664) of white spot syndrome virus, and the bioprobe is an oligonucleotide complementary to the nucleocapsid (VP664) gene.
